(19) United States
(12) Patent Application Publication
(10) Pub. No.: US 2024/0269105 A1
(43) Pub. Date: Aug. 15, 2024

(54) USE OF SGLT-2 INHIBITORS FOR THE PREVENTION AND/OR TREATMENT OF HYPERTENSION IN NON-HUMAN MAMMALS

(71) Applicant: BOEHRINGER INGELHEIM VETMEDICA GMBH, Ingelheim am Rhein (DE)

(72) Inventors: José MATALLO, Frankfurt am Main (DE); Carla KROH, Oestrich-Winkel (DE); Christoph Matthias SCHUMMER, Weilrod (DE)

(21) Appl. No.: 18/578,205

(22) PCT Filed: Jul. 26, 2022
(86) PCT No.: PCT/EP2022/070940

§ 371 Date: Jan. 10, 2024

(30) Foreign Application Priority Data
Jul. 28, 2021 (EP) 21188316.0

(51) Int. Cl. A61K 31/351; A61K 45/06; A61P 9/12

(52) U.S. Cl. CPC A61K 31/351; A61K 45/06; A61P 9/12

(57) ABSTRACT

The present invention is directed to the use of one or more SGLT-2 inhibitors or pharmaceutically acceptable forms thereof for the prophylaxis and/or treatment of hypertension in a non-human mammal, preferably a carnivore, in particular a cat or a dog.

### FIELD OF THE INVENTION

The invention relates to the field of medicine, in particular to the field of veterinary medicine. The invention relates to the use of one or more SGLT-2 inhibitors or pharmaceutically acceptable forms thereof in the prevention and/or treatment of hypertension in a non-human mammal, preferably a carnivore, in particular a cat or a dog.

### BACKGROUND INFORMATION

Systemic hypertension (SHT), which is synonymous with sustained increases in blood pressure (BP), is generally categorized into three types. It can be artificial/transient (stress-induced, situational), secondary to other disease processes that can increase blood pressure (secondary hypertension to e.g. chronic kidney disease) or occur in the absence of other potentially causative disease (idiopathic hypertension).

Hypertension is a common comorbidity in human patients with diabetes type II (up to 86% of this patient cohort present hypertension) and is a major risk factor for the development of cardiac disease, chronic kidney disease, retinal degeneration and subsequent blindness, and stroke. The effects of SGLT-2 inhibition on BP is already recognized in these kinds of patients, however the exact mechanism of action remains elusive.

For example, the clinical trial EMPA-REG BP (N=825) reported that mean 24-hour systolic BP was significantly reduced from baseline to week 12 in both the empagliflozin 10 mg and 25 mg groups compared with the placebo group (placebo-subtracted mean difference up to −4.16 mmHg) in human patients suffering from diabetes mellitus type II. Similar results have been described for other SGLT-2 inhibitors.

Hypertension is a disease of modern civilization and heavily depends on environmental and particularly dietary factors of modern society. Specifically, the renin-angiotensin-aldosterone system has been adapted for sodium retention and has an important role in the pathogenesis of hypertension in human contemporary societies with high dietary salt intake (western diets). Besides, in humans there occurs smoking, lack of physical activity, alcohol abuse, stress, and sleep apnoea. The case is different with non-human mammals, specially carnivores, where hypertension is mostly associated with age and frequently diagnosed in association with other diseases such as chronic kidney disease and hyperthyroidism.

In non-human mammals such as the cat and the dog, the prevalence of hypertension depends on the presence of secondary disease linked to the condition. Chronic kidney disease (CKD), diabetes, hyperthyroidism, or obesity for example, are common causes of high blood pressure with a prevalence ranging from 46% in CKD patients to 15% in diabetic patients. Blood pressure increase is also linked to therapeutic treatments as glucocorticoids, mineralocorticoids, erythropoiesis-stimulating agents, ephedrine, and chronic high-dose sodium chloride. Idiopathic hypertension is chronic kidney disease more common than previously recognized, accounting for approximately 13%-20% of cases in cats and 24% in dogs (from a cohort of 42 animals presenting ocular hypertension, Ref. 1 and Ref. 2).

Clinical signs of hypertension can include sudden blindness, bleeding inside the globe of the eye, and persistently dilated pupils, detached retinas, nervous system signs like depression, head tilt, seizures, disorientation, ataxia, weakness or partial paralysis and nystagmus. Additionally, increased fluid uptake and urinating with the progression of chronic kidney disease, hematuria, epistaxis and heart diseases have been reported.

The diagnosis of hypertension is based on direct blood pressure measurement, with direct arterial catheterization being the gold standard, but for practical reasons indirect BP measuring devices including oscillometry, high-definition oscillometry and doppler sphygmomanometry are preferred.

Typically, treatment starts at the hypertensive stage and the therapeutic goal is to bring blood pressure down to normotensive or pre-hypertensive levels, with the intention to reduce the risk of target organ damage (TOD). In the presence of TOD (e.g., heart and kidney disease) treatment should start immediately, after the initial measurement session. Otherwise, the diagnosis of hypertension should be based on at least two measurement sessions performed on different days.

The ACVIM panel recommends salt dietary restriction in addition to anti-hypertensive agents such as alpha and beta-adrenergic blockers, aldosterone receptor blockers, angiotensin converting enzyme inhibitors (ACEI), angiotensin receptor blockers, and calcium channel blockers depending on the primary cause of hypertension. Additional medications may be required depending on the response to initial therapy. (Ref. 3)

ACEIs are widely used as first-line treatment for SHT in dogs due to the role of the renin-angiotensin-aldosterone system (RAAS) in its development, but they provide an incomplete block of angiotensin II production that can result in poor control of SHT. This phenomenon, called ‘aldosterone breakthrough’, is due to the release of angiotensin II by other sites compared with those regulated by the ACE and independent of the dose of ACEI administered. (Ref. 4)

In humans, a reduction of blood pressure was observed using SGLT2-inhibitors in different clinical trials. However, the direct effect on blood pressure in non-human mammals like felines and canines remains unknown as there are anatomical, physiological, and pathophysiological differences between humans and non-human carnivores.

US 2015/2792977 discloses SGLT2-inhibitors for use in the treatment and/or prevention of a metabolic disorder of an equine animal.

WO 2001/027128 discloses C-aryl glucoside SGLT2-inhibitors.

WO 2003/099836 discloses C-aryl glucoside SGLT2-inhibitors.

WO 2005/012326 discloses novel compounds having inhibitory activity against sodium dependent transporter.

WO 2007/140191 discloses glucose transport inhibitors and methods of use.

WO 2008/042688 discloses inhibitors of sodium glucose co-transporter 2 and methods of their use.

WO 2010/023594 discloses dioxa-bicyclo[3.2.1]octane-2,3,4-triol derivatives.

The International Patent application WO 2015/091313 A1 discloses the use of SGLT-2 inhibitors in or use in the treatment and/or prevention of a metabolic disorder in a feline animal.

The International Patent application WO 2015/110402 A1 discloses the use of SGLT-2 inhibitors in or use in the treatment and/or prevention of a metabolic disorder in a canine animal.

WO 2021/092341 discloses sodium-glucose linked transporter inhibitors for the management of chronic kidney disease, hypertension and heart failure in companion animals.

The International Patent application WO 2021/105152 A1 discloses the use of SGLT-2 inhibitors in the drying-off of non-human mammals.

Dogs often suffer from a type of high blood pressure, which is highly resistant against the treatment with anti-hypertensive agents, in particular ACEIs. This often requires combination of several anti-hypertensive agents (Ref. 3, Ref. 4). The use of anti-hypertensive treatment combinations is also common in cats, where severe hypertension accompanied with the risk of target organ damage requires immediate interference. However, such a combination treatment may cause hypotension, which may deprive the brain and other vital organs of oxygen and nutrients, leading to a life-threatening condition called shock.

Notwithstanding the above disclosures, there is a medical need for the prevention and/or treatment of hypertension in a non-human mammal, preferably a carnivore, in particular a cat or a dog. More specifically, there is a need to develop suitable veterinary prevention(s) and/or treatment(s) for hypertension which are safe and efficacious for non-human animals, including, e.g., cats and dogs.

### SUMMARY OF THE INVENTION

The present invention concerns one or more SGLT-2 inhibitors or pharmaceutically acceptable forms thereof for use in a method of prevention and/or treatment of hypertension in a non-human mammal, preferably a carnivore, in particular a cat or a dog.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the hypertension is selected from the group consisting of: situational hypertension, secondary hypertension, and idiopathic hypertension.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the hypertension is idiopathic hypertension.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In another aspect, the present invention also concerns the one or more SGLT-2 inhibitors or pharmaceutically acceptable forms thereof for the uses as herein disclosed and/or claimed, wherein the non-human mammal, preferably the carnivore, more preferably a cat or a dog suffers from prehypertensive systolic blood pressure (SBP) with low target organ damage (TOD) risk, hypertensive SBP with moderate TOD risk or severely hypertensive SBP with high TOD risk.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In another aspect, the present invention also concerns the one or more SGLT-2 inhibitors or pharmaceutically acceptable forms thereof for the uses as herein disclosed and/or claimed, wherein the one or more SGLT-2 inhibitors are glucopyranosyl-substituted benzene derivatives.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms is thereof for the uses as herein disclosed and/or claimed, wherein the one or more SGLT-2 inhibitors are selected from the group consisting of:
(1) a glucopyranosyl-substituted benzene derivative of the formula (1)

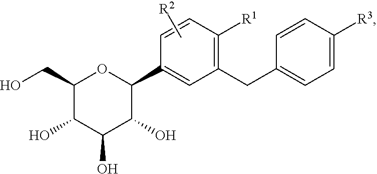

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2):

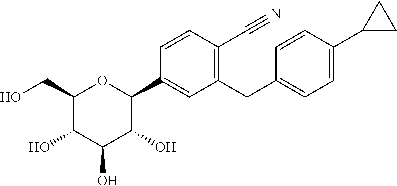 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

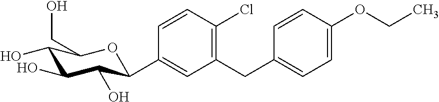

(4) Canagliflozin, represented by formula (4):

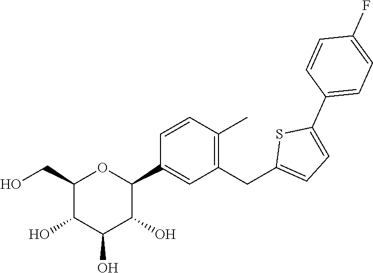 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

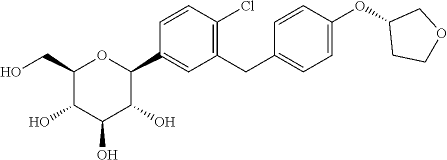 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

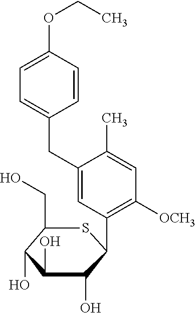

(7) Tofogliflozin, represented by formula (7):

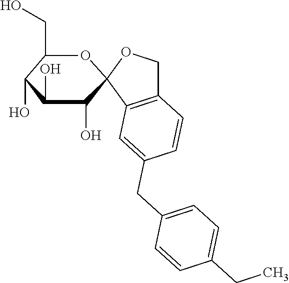

(8) Ipragliflozin, represented by formula (8):

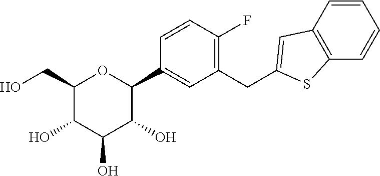 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

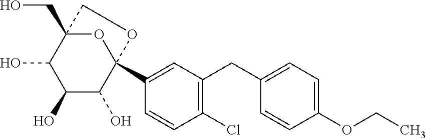 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

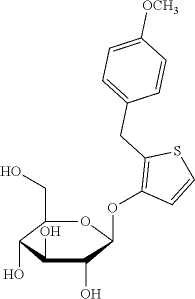

(11) Remogliflozin, represented by formula (11):

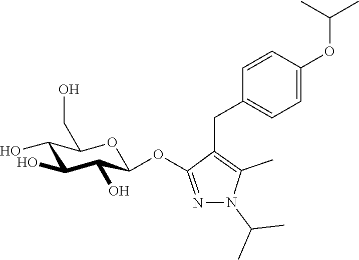

(11A) Remogliflozin etabonate, represented by formula (11A):

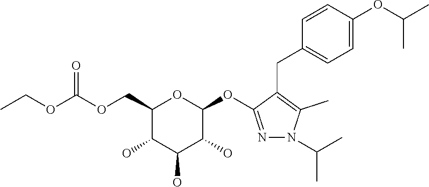 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

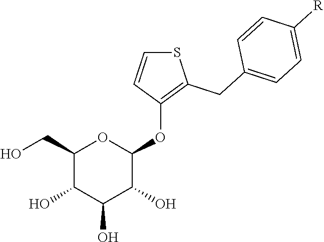

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

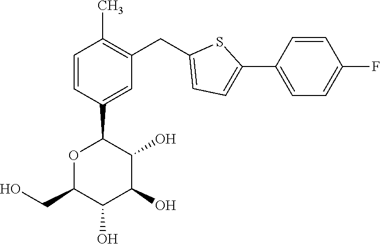 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

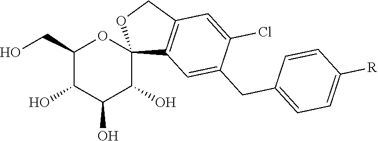

wherein R denotes methoxy, trifluoromethoxy, ethoxy, ethyl, isopropyl or tert. butyl;
(15) a pyrazole-O-glucoside derivative of the formula (15)

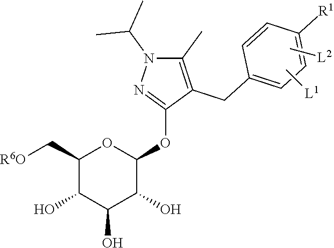

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

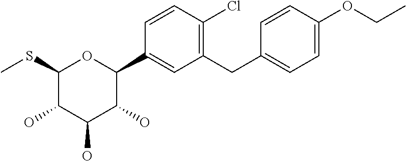 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

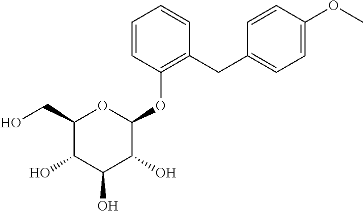 [SMILES: COc1ccc(Cc2ccccc2O[C@@H]2O[C@H](CO)[C@@H](O)[C@H](O)[C@H]2O)cc1]

(18) a compound represented by formula (18):

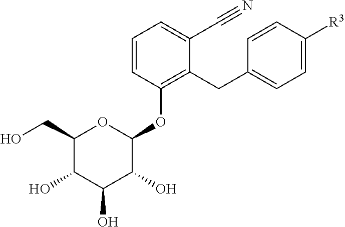

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

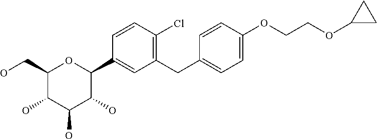 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

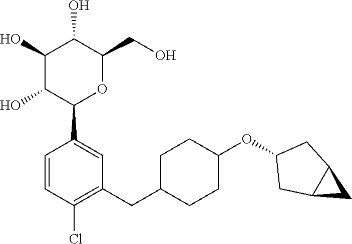 [SMILES: OC[C@H]1O[C@@H](c2ccc(Cl)c(CC3CCC(O[C@H]4C[C@@H]5C[C@@H]5C4)CC3)c2)[C@H](O)[C@@H](O)[C@@H]1O]

(21) Rongliflozin, represented by formula (21):

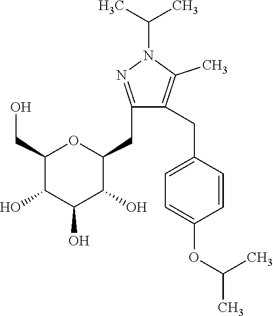 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the pharmaceutically acceptable form thereof is a crystalline complex between the one or more SGLT2 inhibitors and one or more amino acids, preferably proline, more preferably L-proline; and most preferably is co-crystal of the one or more SGLT2 inhibitors, L-proline and crystalline water.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the a non-human mammal, preferably the carnivore, in particular a cat or a dog, is a carnivore patient in need of such prevention and/or treatment; and preferably is a cat patient or a dog patient in need of such prevention and/or treatment, more preferably a non-diabetic cat patient or a non-diabetic dog patient in need of such prevention and/or treatment.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are administered orally, parenterally, intravenously, subcutaneously or intramuscularly, preferably orally.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are to be administered at a dose of 0.01 mg/kg bodyweight to 10 mg/kg bodyweight, preferably at a dose of 0.01 mg/kg bodyweight to 5 mg/kg bodyweight, more preferably at a dose of 0.01 mg/kg bodyweight to 4 mg/kg bodyweight, even more preferably at a dose of 0.01 mg/kg bodyweight to 3 mg/kg bodyweight, even more preferably at a dose of 0.01 mg/kg bodyweight to 2 mg/kg bodyweight, even more preferably at a dose of 0.01 mg/kg bodyweight to 1 mg/kg bodyweight, even more preferably at a dose of 0.02 mg/kg bodyweight to 1 mg/kg bodyweight, most preferably at a dose of 0.04 mg/kg bodyweight to 1 mg/kg bodyweight.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein such one or more SGLT2 inhibitors or pharmaceutically acceptable forms thereof is to be administered once or twice per day.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors is velagliflozin, which is to be administered as a single SGLT-2 inhibitor, preferably orally, more preferably once or twice per day at a dose of 0.01 mg/kg bodyweight to 1 mg/kg bodyweight, even more preferably at a dose of 0.02 mg/kg bodyweight to 1 mg/kg bodyweight, even more preferably at a dose of 0.04 mg/kg bodyweight to 1 mg/kg bodyweight.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are to be administered before, after or concomitantly with administering one or more other active pharmaceutical ingredients, preferably diuretics, such as furosemide, torasemide or spironolactone; beta-blockers, such as atenolol or propranolol; calcium-channel blockers, such as diltiazem; ACE inhibitors, such as benazepril, ramipril or enalapril; angiotensin receptors blockers, such as telmisartan; antiarrhythmic agents, such as flecainide; platelet agglutination inhibitors, such as clopidogrel; nonsteroidal anti-inflammatory drugs (NSAIDs), such as aspirin; anticoagulants, such as coumarins (vitamin K antagonists), (low molecular weight) heparin, synthetic pentasaccharide inhibitors of factor Xa, as well as direct factor Xa inhibitors and/or direct thrombin inhibitors; and/or calcium-channel sensitizers and/or positive inotropes, such as pimobendan and/or digitalis alkaloids.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the systolic blood pressure (SBP) value measured for the non-human mammal, preferably carnivore, more preferably a cat or a dog, in need thereof is decreased after the period of the treatment by at least 5 mmHg, preferably by at least 10 mmHg, more preferably by at least 20 mmHg, in particular by 5 to 100 mmHg, more preferably 5 to 50 mmHg, most preferably by 10 to 50 mmHg, in relation to the baseline SBP value measured for the non-human mammal, preferably carnivore, more preferably a cat or a dog, prior to the period of treatment.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the method comprises measurement of the SBP and optionally identification of TOD followed by administration of a therapeutically effective amount of the one or more SGLT-2 inhibitors or pharmaceutically acceptable forms thereof to the non-human mammal in need thereof, preferably carnivore, more preferably a cat or a dog, wherein the therapeutically effective amount of the one or more SGLT-2 inhibitors or pharmaceutically acceptable forms thereof is administered in a daily dosage amount that may be varied over a treatment period depending on subsequent measurements of the SBP.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the hypertension is non-refractory to the treatment with ACE inhibitors in the non-human mammal to be treated, preferably the carnivore to be treated, in particular the cat or dog to be treated, most preferably the dog to be treated.

A corresponding method of preventing and/or treating hypertension in a non-human mammal, preferably a carnivore, in particular a cat or a dog, comprising administering one or more SGLT-2 inhibitors to such non-human mammal, preferably such carnivore, in particular such cat or such dog, as herein disclosed and/or claimed, as well as the corresponding use of one or more SGLT-2 inhibitors for the preparation of a medicament for the prevention and/or treatment of hypertension in a non-human mammal, preferably a carnivore, in particular a cat or a dog, as herein disclosed and/or claimed, are also intended to be comprised by the present invention.

The present invention further concerns a pharmaceutical composition comprising one or more SGLT2 inhibitors or pharmaceutically acceptable forms thereof as herein disclosed and/or claimed for the uses/methods as herein disclosed and/or claimed.

### DETAILED DESCRIPTION OF THE INVENTION

Before the embodiments of the present invention are described in further detail, it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In the course of the present invention, the terms “carnivore” and “predominantly carnivorous non-human mammal” are used interchangeably. In one preferred embodiment the carnivore is a predominantly carnivorous non-human mammal, more preferred a canine, in particular a dog, and/or a feline, in particular a cat. In another preferred embodiment, the “non-human mammal” is selected from the group consisting of: bovine, canine, caprine, equine, feline, lagomorphs, ovine, porcine, rodent; more preferably is selected from the group consisting of: cattle, cow, dog, goat, horse, pony, donkey, cat, sheep, pig, rabbit, rat, mouse; even more preferably selected from the group consisting of: canine and/or feline; most preferably selected from the group consisting of: dog and cat. Mammals are a class of vertebrate animals, whose females are characterized by the possession of mammary glands while both males and females are characterized by sweat glands, hair, three middle ear bones used in hearing, and a neocortex region in the brain. Within this class the placentals are preferred, which are characterized using a placenta during gestation.

Mammals can further be divided with respect to their feeding. Some mammals feed on animal prey—this is a carnivorous diet (and includes insectivorous diets). Other mammals, called herbivores, eat plants. An omnivore eats both prey and plants. Carnivorous mammals have a simple digestive tract, because the proteins, lipids, and minerals found in meat require little in the way of specialized digestion. Plants, on the other hand, contain complex carbohydrates, such as cellulose. The digestive tract of an herbivore is therefore host to bacteria that ferment these substances and make them available for digestion. The present invention is especially designed for carnivores and predominantly carnivorous non-human mammals. Such mammals include especially all feliforms, such as domestic cats or big cats, and most caniforms, such as the dogs, wolves and foxes. Due to the economic importance of companion animals in modern life, the present invention is especially designed for dogs and/or for cats.

The non-human mammals to be treated with one or more SGLT-2 inhibitors according to the invention are preferably domesticated carnivores such as cats or dogs of any breed including any kind of mongrel. Depending on the size of the breed or mongrel they may suffer from hypertension at an any age, but more frequently at an age of 5 years or more, preferably from 7 to 18 years, in particular from 10 to 16 years. Small breeds will as a rule suffer at a later age, preferably from 12 to 18, from this disease than big ones, which may be affected at an age of 10 to 16 years.

As used herein, the term “hypertension” refers to an elevated pressure of the blood against the walls of arteries during the time the heart contracts and empties itself of blood as well as during the time the heart relaxes and fills with blood and in. The term embraces systemic hypertension and idiopathic hypertension.

The term “systemic hypertension” or abbreviated SHT is applied to sustained increases in systolic blood pressure (SBP>140 mmHg), and generally can be categorized into 1 of 3 types: (i) it may be caused by environmental or situational stressors, (ii) it may occur in association with other disease processes that increase BP (ie, secondary hypertension), or (iii) it may occur in the absence of other potentially causative disease processes (ie, idiopathic hypertension).

“SHT in cats and dogs is classified based on the risk of target organ damage (TOD) according to the ACVIM consensus statement (Ref. 3), as follows:
Normotensive (minimal TOD risk) systolic BP<140 mm Hg
Pre-hypertensive (low TOD risk) systolic BP 140-159 mmHg
Hypertensive (moderate TOD risk) systolic BP 160-179 mm Hg
Severely hypertensive (high TOD risk) systolic BP>180 mm Hg.

As used herein, the term “non-refractory to the treatment with ACE inhibitors (ACEI)” refers to non-human mammals, preferably carnivores, in particular cats or dogs suffering from hypertension, which can be treated with an ACEI, but with less efficacy than an SGLT-2 inhibitor. To the contrary the high values of sustained systolic arterial blood pressure (SBP) of non-human mammals that are refractory to ACEIs cannot be lowered with the aid of ACEIs.

In the non-refractory sub-population of non-human mammals the efficacy of treatment with an ACEI is 10%, 15%, 20%, 25%, 30%, 35%, 40%, 45%, 50%, more than 50%, more than 60%, or more than 70% less effective than an SGLT-2 inhibitor for lowering their SBP values.

In a preferred embodiment the one or more SGLT-2 inhibitor according to the invention relates to the treatment and/or prevention of the non-refractory subpopulation of dogs.

SGLT-2 inhibitors for use according to the invention include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Moreover, the one or more SGLT-2 inhibitors for use according to the invention may be selected from the group consisting of the following compounds or pharmaceutically acceptable forms thereof:
(1) a glucopyranosyl-substituted benzene derivative of the formula (1)

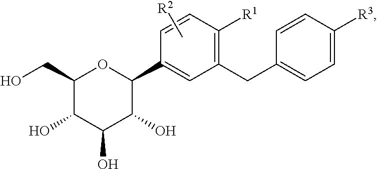

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2):

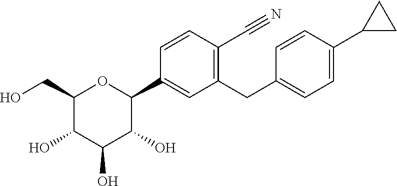 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

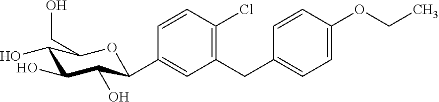

(4) Canagliflozin, represented by formula (4):

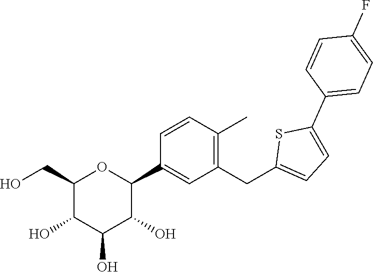 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

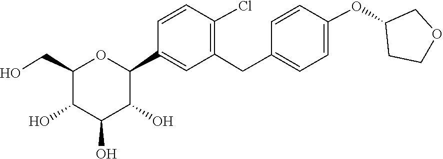 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

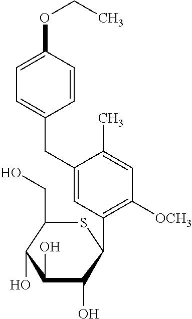

(7) Tofogliflozin, represented by formula (7):

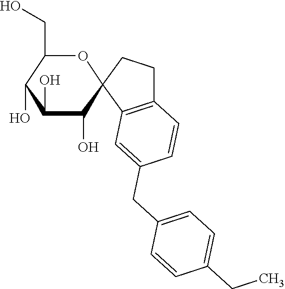

(8) Ipragliflozin, represented by formula (8):

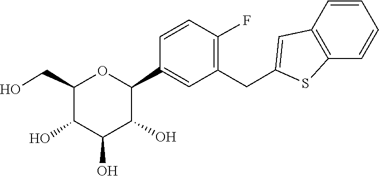 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

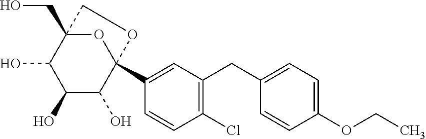 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

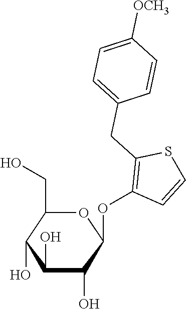

(11) Remogliflozin, represented by formula (11):

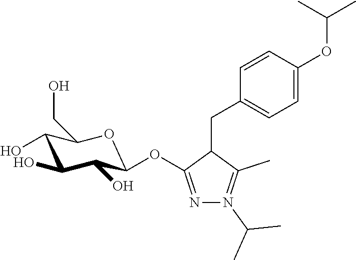

(11A) Remogliflozin etabonate, represented by formula (11A):

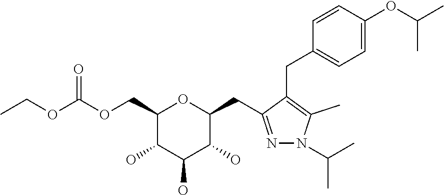 [SMILES: CCOC(=O)OC[C@H]1O[C@@H](Cc2nn(C(C)C)c(C)c2Cc2ccc(OC(C)C)cc2)[C@H](O)[C@@H](O)[C@@H]1O]

(12) a thiophene derivative of the formula (12)

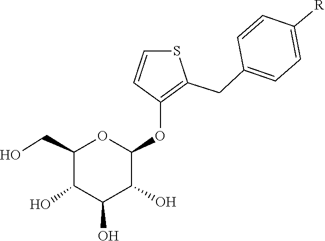

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

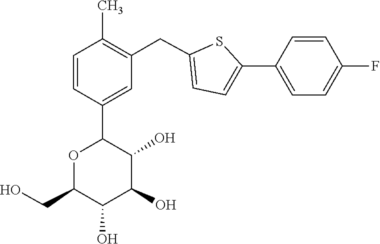 [SMILES: Cc1ccc(C2O[C@H](CO)[C@@H](O)[C@H](O)[C@H]2O)cc1Cc1ccc(-c2ccc(F)cc2)s1]

(14) a spiroketal derivative of the formula (14):

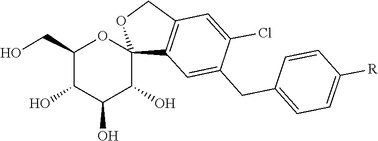

wherein R denotes methoxy, trifluoromethoxy, ethoxy, ethyl, isopropyl or tert. butyl;
(15) a pyrazole-O-glucoside derivative of the formula (15)

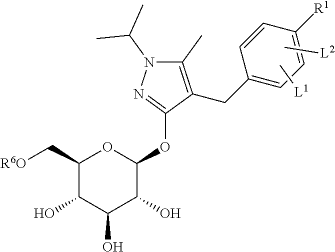

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

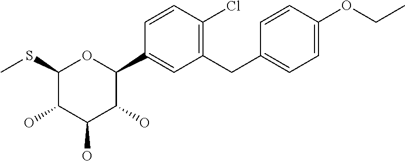 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

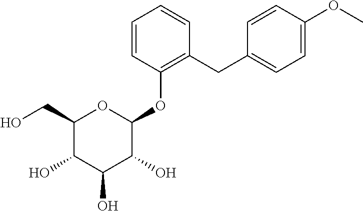 [SMILES: COc1ccc(Cc2ccccc2O[C@@H]2O[C@H](CO)[C@@H](O)[C@H](O)[C@H]2O)cc1]

(18) a compound represented by formula (18):

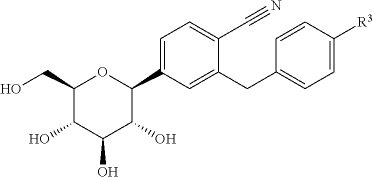

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

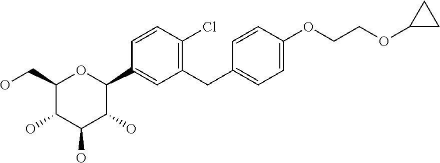 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

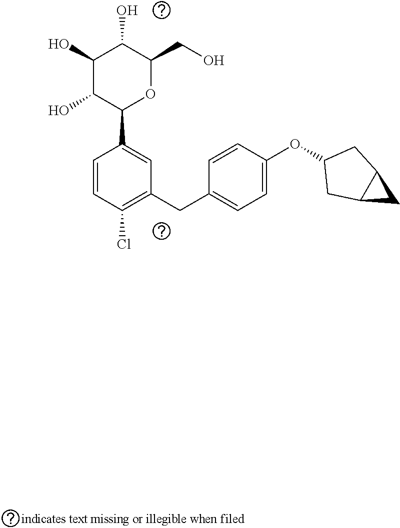

(21) Rongliflozin,

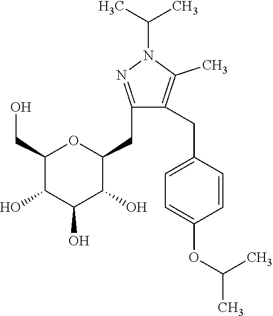 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin.

The term “velagliflozin” as employed herein refers to velagliflozin of the above structure (2) as well as pharmaceutically acceptable forms thereof, including hydrates and solvates thereof, and crystalline forms thereof. The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof. The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof. The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e. sotagliflozin, and its manufacture are described for example in WO 2008/042688 or WO 2009/014970.

Preferred SGLT-2 inhibitors are glucopyranosyl-substituted benzene derivatives. Optionally, one or more hydroxyl groups of the glucopyranosyl group in such one or more SGLT-2 inhibitors may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

More preferred are glucopyranosyl-substituted benzonitrile derivatives of formula (1) as disclosed herein above. Yet more preferred are glucopyranosyl-substituted benzonitrile derivatives of formula (18):

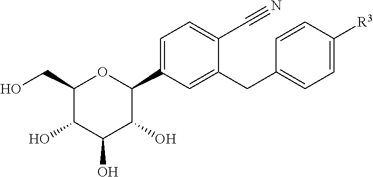

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Preferably, such SGLT-2 inhibitor is velaglifozin as shown in formula (2). Optionally, one or more hydroxyl groups of the β-D-glucopyranosyl group of velagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in a preferred embodiment, the at least one SGLT-2 inhibitor according to the present invention is a glucopyranosyl-substituted benzene derivative SGLT-2 inhibitor, preferably a SGLT-2 inhibitor of formula (1), more preferably of formula (18), or yet more preferably of formula (2), i.e., velagliflozin, in each case as defined herein above.

Herein, references to SGLT-2 inhibitors and/or their use according to the invention encompass pharmaceutically acceptable forms of the SGLT-2 inhibitors, unless otherwise stated.

According to the invention, any pharmaceutically acceptable form of the SGLT-2 inhibitor, e.g., of formula (1), preferably formula (18), more preferably formula (2), may be used. E.g., a crystalline form may be used. Prodrug forms are also encompassed by the present invention.

Prodrug forms may include, e.g., esters and/or hydrates. The term “prodrug” is also meant to include any covalently bonded carrier, which releases the active compound of the invention in vivo when the prodrug is administered to a non-human mammalian subject. Prodrugs of a compound of the invention may be prepared by modifying functional groups present in the compound of the invention in such a way that the modifications are cleaved, either in routine manipulation or in vivo, to the parent compound of the invention.

Crystalline forms for use according to the invention include a complex of an SGLT-2 inhibitor with one or more amino acids (see e.g. WO 2014/016381)—so-called co-crystals. An amino acid for such use may be a natural amino acid. The amino acid may be a proteogenic amino acid (including L-hydroxyproline), or a non-proteogenic amino acid. The amino acid may be a D- or an L-amino acid. In some preferred embodiments, the amino acid is proline (L-proline and/or D-proline, preferably L-proline). E.g., a crystalline complex/co-crystal of velagliflozin with proline (e.g. L-proline) and crystalline water is preferred.

Thus, herein is disclosed a crystalline complex/co-crystal between one or more natural amino acids and an SGLT-2 inhibitor, e.g., a crystalline complex/co-crystal between one or more natural amino acids and a glucopyranosyl-substituted benzene derivative SGLT-2 inhibitor, preferably a SGLT-2 inhibitor of formula (1), more preferably of formula (18) or yet more preferably of formula (2) (velagliflozin).

A certain pharmaceutical activity is the basic prerequisite to be fulfilled by a pharmaceutically active agent before it is approved as a medicament on the market. However, there are a variety of additional requirements a pharmaceutically active agent has to comply with. These requirements are based on various parameters, which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions, which contain, in addition to the actual active substance, breakdown products thereof, for example. In such cases, the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g. by grinding. Since breakdown of the pharmaceutically active substance as a side effect of the grinding (or micronizing) has to be avoided as far as possible, in spite of the hard conditions required during the process, it is essential that the active substance should be highly stable throughout the grinding process. Only if the active substance is sufficiently stable during the grinding process is it possible to produce a homogeneous pharmaceutical formulation, which always contains the specified amount of active substance in a reproducible manner.

Another problem, which may arise in the grinding process for preparing the desired pharmaceutical formulation, is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament; the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance because of the increased weight caused by the uptake of water. Pharmaceutical compositions with a tendency to absorb moisture have to be protected from moisture during storage, e.g. by the addition of suitable drying agents or by storing the drug in an environment where it is protected from moisture. Preferably, therefore, a pharmaceutically active substance should be at best slightly hygroscopic.

Further lore, the availability of a well-defined crystalline form allows the purification of the drug substance by recrystallization.

Apart from the requirements indicated above, it should be generally borne in mind that any change to the solid state of a pharmaceutical composition, which is capable of improving its physical and chemical stability, gives a significant advantage over less stable forms of the same medicament.

A crystalline complex/co-crystal between a natural amino acid and an SGLT-2 inhibitor (e.g. a glucopyranosyl-substituted benzene derivative or a SGLT-2 inhibitor of formula (1), or formula (18) or, particularly, of formula (2), i.e. velaglilfozin) fulfills important requirements mentioned hereinbefore.

SGLT-2 inhibitors for use according to the invention may be prepared as pharmaceutical compositions. They may be prepared as solid or as liquid formulations. In either case, they are preferably prepared for oral administration, preferably in liquid form for oral administration (see e.g. WO 2017/032799). The SGLT-2 inhibitors may, however, also be prepared, e.g., for parenteral administration. Solid formulations include tablets, granular forms, and other solid forms such as suppositories. Among solid formulations, tablets and granular forms are preferred.

Pharmaceutical compositions within the meaning of the present invention may comprise an SGLT-2 inhibitor according to the present invention and one or more excipients. Any excipient that allows for, or supports, the intended medical effect may be used. Such excipients are available to the skilled person. Useful excipients are for example anti-adherents (used to reduce the adhesion between the powder (granules) and the punch faces and thus prevent sticking to tablet punches), binders (solution binders or dry binders that hold the ingredients together), coatings (to protect tablet ingredients from deterioration by moisture in the air and make large or unpleasant-tasting tablets easier to swallow), disintegrants (to allow the tablet to break upon dilution), fillers, diluents, flavours, colours, glidants (flow regulators—to promote powder flow by reducing interparticle friction and cohesion), lubricants (to prevent ingredients from clumping together and from sticking to the tablet punches or capsule filling machine), preservatives, sorbents, sweeteners etc.

Formulations according to the invention, e.g. solid formulations, may comprise carriers and/or disintegrants selected from the group of sugars and sugar alcohols, e.g. mannitol, lactose, starch, cellulose, microcrystalline cellulose and cellulose derivatives, e.g. methylcellulose, and the like.

Manufacturing procedures for formulations suitable for canines are known to the person skilled in the art, and for solid formulations comprise, e.g., direct compression, dry granulation and wet granulation. In the direct compression process, the active ingredient and all other excipients are placed together in a compression apparatus that is directly applied to press tablets out of this material. The resulting tablets can optionally be coated afterwards in order to protect them physically and/or chemically, e.g. by a material known from the state of the art.

A unit for administration, e.g. a single liquid dose or a unit of a solid formulation, e.g. a tablet, may comprise 0.1 mg to 10 mg, or e.g. 0.3 mg to 1 mg, 1 mg to 3 mg, 3 mg to 10 mg; or 5 to 2500 mg, or e.g. 5 to 2000 mg, 5 mg to 1500 mg, 10 mg to 1500 mg, 10 mg to 1000 mg, or 10-500 mg of an SGLT-2 inhibitor for use according to the invention. As the skilled person would understand, the content of the SGLT-2 inhibitor in a solid formulation, or any formulation as disclosed herein for administration to a non-human mammal, preferably a carnivore, in particular a feline or a canine animal, may be increased or decreased as appropriate in proportion to the body weight of the canine animal to be treated.

In one embodiment, a pharmaceutical composition for use according to the invention is designed for oral or parenteral administration, preferably for oral administration. Especially the oral administration is ameliorated by excipients, which modify the smell and/or haptic properties of the pharmaceutical composition for the intended patient, e.g. as described.

When the SGLT-2 inhibitor for use according to the invention is formulated for oral administration, it is preferred that excipients confer properties, e.g., palatability and/or chewability that render the formulation suitable for administration to a non-human mammal, preferably a carnivore, in particular a feline or a canine animal.

Also preferred are liquid formulations. Liquid formulations may be, e.g., solutions, syrups or suspensions. They may be administered directly to the canines or may be mixed with the food and/or drink (e.g. drinking water, or the like) of the canine animal. One advantage of a liquid formulation (similar to a formulation in granular form), is that such a dosage fount allows precise dosing. For example, the SGLT-2 inhibitor may be dosed precisely in proportion to the body mass of a non-human mammal, preferably a carnivore, in particular a feline or a canine animal. Typical compositions of liquid formulations are known to the person skilled in the art.

A practitioner skilled in the art can determine suitable doses for the uses of the present invention. Preferred units dosing units include mg/kg bodyweight, i.e., mg SGLT-2 inhibitor per body mass of the non-human mammal. An SGLT-2 inhibitor of the invention may, e.g., be administered in doses of 0.01-10 mg/kg bodyweight per day, e.g. 0.01-5 mg/kg bodyweight per day, e.g. 0.01-4 mg/kg bodyweight per day, e.g. 0.01-3 mg/kg bodyweight per day, e.g. 0.01-2 mg/kg bodyweight per day, e.g. 0.01-1.5 mg/kg bodyweight per day, e.g., 0.01-1 mg/kg bodyweight per day, e.g. 0.01-0.75 mg/kg bodyweight per day, e.g. 0.01-0.5 mg/kg bodyweight per day, e.g. 0.01-0.4 mg/kg bodyweight per day; or 0.1 to 3.0 mg/kg bodyweight per day, preferably from 0.2 to 2.0 mg/kg bodyweight per day, more preferably from 0.1 to 1 mg/kg bodyweight per day or from 0.5 to 1 mg/kg bodyweight per day. In another preferred embodiment, the dose is 0.01-1 mg/kg bodyweight per day, preferably 0.02-1 mg/kg bodyweight per day, more preferably 0.04-0.1 mg/kg bodyweight per day, e.g. 0.03-1 mg/kg bodyweight per day.

In a specific embodiment, the dose is 1 mg/kg bodyweight per day in a feline patient and 0.3 or 1 mg/kg bodyweight per day in a canine patient.

A practitioner skilled in the art is able to prepare an SGLT-2 inhibitor of the invention for administration according to a desired dose.

### EXAMPLES

The following examples serve to further illustrate the present invention; but the same should not be construed as a limitation of the scope of the invention disclosed herein.

### Example 1 Clinical Field Study (“Cats”)

In a clinical field study, the effect of velagliflozin on systolic blood pressure administered to cats (n=5-10) previously diagnosed with high systolic blood pressure, is evaluated over a period of 1 month. Systolic blood pressure measurements are performed in accordance with the ACVIM consensus statement (Ref. 3). Systolic blood pressure is measured before treatment start and during weekly consecutive visits (4 weeks of treatment). The study is an open label study (unmasked). The data of this field trial confirm an average reduction of systolic blood pressure of about 5-10 mmHg.

### Example 2 Clinical Field Study (“Dogs”)

In a clinical field study, the effect of velagliflozin on systolic blood pressure administered to dogs (n=5-10) previously diagnosed with high systolic blood pressure, is evaluated over a period of 1 month. Systolic blood pressure measurements are performed in accordance with the ACVIM consensus statement (Ref. 3). Systolic blood pressure is measured before treatment start and during weekly consecutive visits (4 weeks of treatment). The study is an open label study (unmasked). The data of this field trial confirm an average reduction of systolic blood pressure of about 5-10 mmHg.

### References

The following publications are hereby incorporated by reference in their entirety as if each individual publication is specifically and individually indicated to be incorporated by reference. In case of conflict, the present application, including any definitions herein, will control.
(1) Ref. 1: Jepson R E. Feline systemic hypertension: classification and pathogenesis. J Feline Med Surg. 2011;13:25-34
(2) Ref. 2: Ocular lesions associated with systemic hypertension in dogs: 65 cases (2005-2007); Nicole L. LeBlanc, DVM; Rebecca L. Stepien, DVM, MS, DACVIM; Ellison Bentley, DVM, DACVO; Journal of the American Veterinary Association. Apr. 1, 2011, Vol. 238, No. 7, Pages: 915-921
(3) Ref. 3: Mark J. Acierno; Scott Brown, Amanda E. Coleman; Rosanne E. Jepson; Mark Papich; Rebecca L. Stepien and Harriet M. Syme. ACVIM consensus statement: Guidelines for the identification, evaluation, and management of systemic hypertension in dogs and cats. J Vet Intern Med. 2018:32:1803-1822.
(4) Ref. 4: Ames M K, Atkins C E, Lee S, et al. Effects of high doses of enalapril and benazepril on the pharmacologically activated renin-angiotensin-aldosterone system in clinically normal dogs. Am J Vet Res 2015; 76:1041-50.
(5) US 2015/2792977
(6) WO 2001/027128
(7) WO 2003/099836
(8) WO 2005/012326
(9) WO 2007/140191
(10) WO 2008/042688
(11) WO 2010/023594
(12) WO 2015/091313
(13) WO 2015/110402
(14) WO 2021/092341
(15) WO 2021/105152

1. One or more SGLT-2 inhibitors or pharmaceutically acceptable forms thereof for use in a method of prevention and/or treatment of hypertension in a non-human mammal, preferably a carnivore, more preferably a cat or a dog.

2. The one or more SGLT-2 inhibitors or pharmaceutically acceptable forms thereof for the use according to claim 1, wherein the hypertension is selected from the group consisting of: situational hypertension, secondary hypertension and idiopathic hypertension.

3. The one or more SGLT-2 inhibitors or pharmaceutically acceptable forms thereof for the use according to claim 2, wherein the secondary hypertension is selected from the group consisting of hypertension associated with chronic kidney disease (CKD), diabetes, obesity, heart disease, endocrine disease, such as Cushing's disease, hyperthyroidism, acromegaly, and elevated blood pressure (BP) induced by medicaments, preferably by glucocorticoids, mineralocorticoids, erythropoiesis-stimulating agents, ephedrine and/or high dose sodium chloride.

4. The one or more SGLT-2 inhibitors or pharmaceutically acceptable forms thereof for the use according to claim 2, wherein the hypertension is idiopathic hypertension.

5. The one or more SGLT-2 inhibitors or pharmaceutically acceptable forms thereof for the use according to any one of claims 1 to 4, wherein the non-human mammal, preferably the carnivore, more preferably a cat or a dog, suffers from prehypertensive systolic blood pressure (SBP) with low target organ damage (TOD) risk, hypertensive SBP with moderate TOD risk or severely hypertensive SBP with high TOD risk.

6. The one or more SGLT-2 inhibitors or pharmaceutically acceptable forms thereof for the use according to any one of claims 1 to 5, wherein the one or more SGLT-2 inhibitors are glucopyranosyl-substituted benzene derivatives.

7. The one or more SGLT-2 inhibitors or pharmaceutically acceptable forms is thereof for the use according to any one of claims 1 to 6, wherein the one or more SGLT-2 inhibitors are selected from the group consisting of:
(1) a glucopyranosyl-substituted benzene derivative of the formula (1) 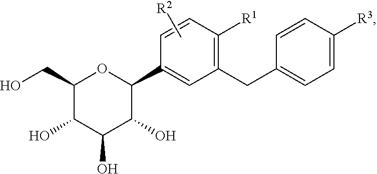
wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2): 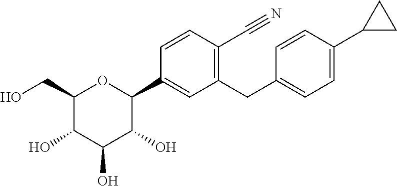 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by formula (3): 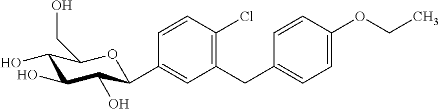
(4) Canagliflozin, represented by formula (4): 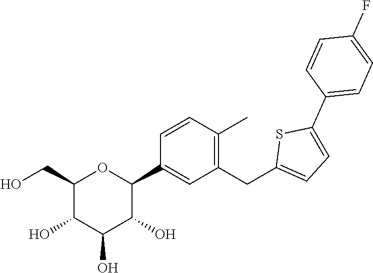 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by formula (5): 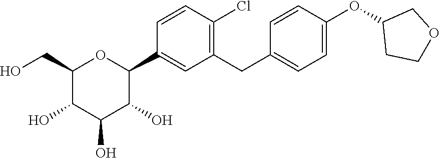
(6) Luseogliflozin, represented by formula (6): 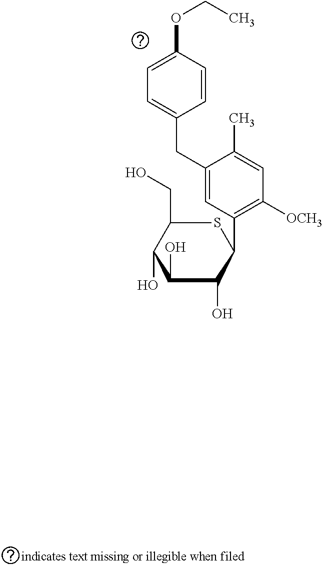
(7) Tofogliflozin, represented by formula (7): 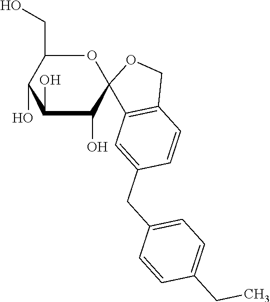
(8) Ipragliflozin, represented by formula (8): 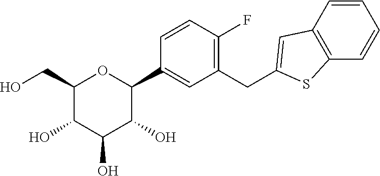 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by formula (9): 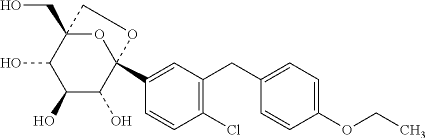 [SMILES: CCOc1ccc(Cc2cc([C@]34OC[C@](CO)(O3)[C@@H](O)[C@H](O)[C@H]4O)ccc2Cl)cc1]
(10) Atigliflozin, represented by formula (10): 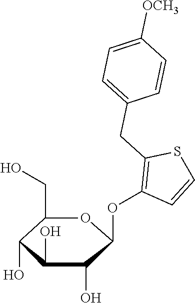
(11) Remogliflozin, represented by formula (11): 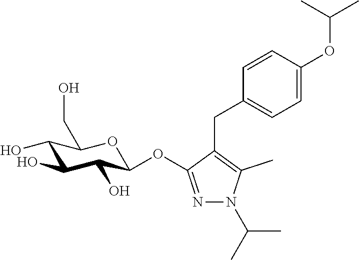
(11A) Remogliflozin etabonate, represented by formula (11A): 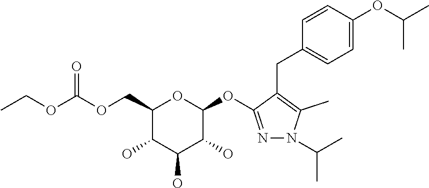 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative of the formula (12) 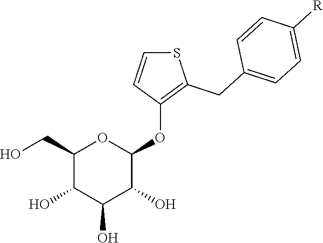
wherein R denotes methoxy or trifluoromethoxy;
(13) 1-(β-D-glucopyranosyl)-4-methyl-3-[5-(4-fluorophenyl)-2-thienylmethyl]benzene, represented by formula (13); 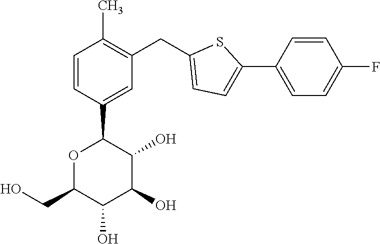 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative of the formula (14): 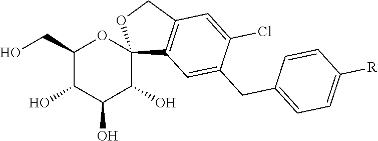
wherein R denotes methoxy, trifluoromethoxy, ethoxy, ethyl, isopropyl or tert. butyl;
(15) a pyrazole-O-glucoside derivative of the formula (15) 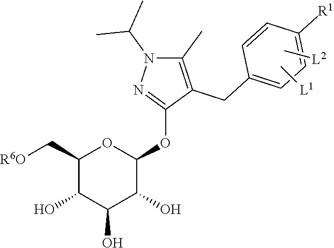
wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16): 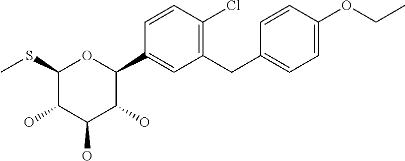 [SMILES: CCOc1ccc(Cc2cc([C@@H]3O[C@H](SC)[C@@H](O)[C@H](O)[C@H]3O)ccc2Cl)cc1]
(17) Sergliflozin, represented by formula (17): 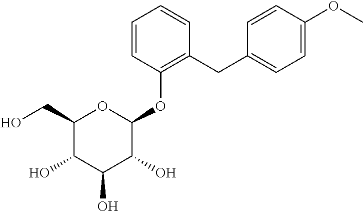 [SMILES: COc1ccc(Cc2ccccc2O[C@@H]2O[C@H](CO)[C@@H](O)[C@H](O)[C@H]2O)cc1]
(18) a compound represented by formula (18): 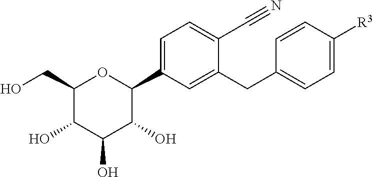
wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19): 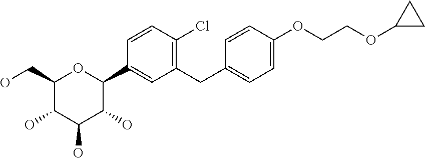 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by formula (20): 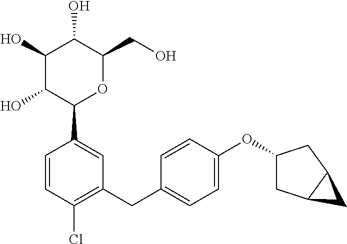 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin represented by formula (21), 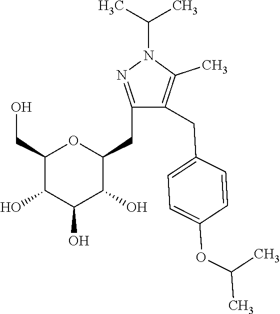 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin.

8. The one or more SGLT-2 inhibitors or pharmaceutically acceptable forms thereof for the use according to any one of claims 1 to 7, wherein the pharmaceutically acceptable form thereof is a crystalline complex between the one or more SGLT2 inhibitors and one or more amino acids, preferably proline, more preferably L-proline; and most preferably is co-crystal of the one or more SGLT2 inhibitors, L-proline and crystalline water.

9. The one or more SGLT-2 inhibitors or pharmaceutically acceptable forms thereof for the use according to any one of claims 1 to 8, wherein the non-human mammal, preferably carnivore, more preferably a cat or a dog, is a non-diabetic non-human mammal patient, preferably a non-diabetic carnivore patient in need of such prevention and/or treatment; more preferably a non-diabetic cat patient or a non-diabetic dog patient in need of such prevention and/or treatment.

10. The one or more SGLT-2 inhibitors or pharmaceutically acceptable forms thereof for the use according to any one of claims 1 to 9, wherein the one or more SGLT-2 inhibitors are administered orally, parenterally, intravenously, subcutaneously, or intramuscularly, preferably orally.

11. The one or more SGLT-2 inhibitors or pharmaceutically acceptable forms thereof for the use according to any one of claims 1 to 10, wherein the one or more SGLT-2 inhibitors are to be administered at a dose of 0.01 mg/kg bodyweight to 10 mg/kg bodyweight, preferably at a dose of 0.01 mg/kg bodyweight to 5 mg/kg bodyweight, more preferably at a dose of 0.01 mg/kg bodyweight to 4 mg/kg bodyweight, even more preferably at a dose of 0.01 mg/kg bodyweight to 3 mg/kg bodyweight, even more preferably at a dose of 0.01 mg/kg bodyweight to 2 mg/kg bodyweight, even more preferably at a dose of 0.01 mg/kg bodyweight to 1 mg/kg bodyweight, even more preferably at a dose of 0.02 mg/kg bodyweight to 1 mg/kg bodyweight, most preferably at a dose of 0.04 mg/kg bodyweight to 1 mg/kg bodyweight.

12. The one or more SGLT-2 inhibitors or pharmaceutically acceptable forms thereof for the use according to any one of claims 1 to 11, wherein such one or more SGLT-2 inhibitors or pharmaceutically acceptable forms thereof is to be administered once or twice per day.

13. The one or more SGLT-2 inhibitors or pharmaceutically acceptable forms thereof for the use according to any one of claims 2 to 12, wherein the one or more SGLT-2 inhibitors is velagliflozin, which is to be administered as a single SGLT-2 inhibitor, preferably orally, more preferably once or twice per day at a dose of 0.01 mg/kg bodyweight to 1 mg/kg bodyweight, even more preferably at a dose of 0.02 mg/kg bodyweight to 1 mg/kg bodyweight, even more preferably at a dose of 0.04 mg/kg bodyweight to 1 mg/kg bodyweight.

14. The one or more SGLT-2 inhibitors or pharmaceutically acceptable forms thereof for the use according to any one of claims 1 to 13, wherein the one or more SGLT-2 inhibitors are to be administered before, after or concomitantly with administering one or more other active pharmaceutical ingredients, preferably another SGLT-2 inhibitor or a pharmaceutically acceptable form thereof; one or more diuretics, such as hydrochlorothiazide, furosemide, torasemide or spironolactone; one or more alpha blockers, such as prazosin, phenoxybenzamine, acepromazine; one or more beta-blockers, such as atenolol or propranolol; one or more calcium-channel blockers, such as amlodipine and diltiazem; one or more ACE inhibitors, such as benazepril, ramipril or enalapril; one or more angiotensin receptors blockers, such as telmisartan; one or more antiarrhythmic agents, such as flecainide; one or more platelet agglutination inhibitors, such as clopidogrel; one or more nonsteroidal anti-inflammatory drugs (NSAIDs), such as aspirin; one or more anticoagulants, such as Coumarins (vitamin K antagonists), (low molecular weight) heparin, synthetic pentasaccharide inhibitors of factor Xa, one or more direct factor Xa inhibitors; one or more direct thrombin inhibitors; one or more calcium-channel sensitizers, one or more positive inotropes, such as pimobendan; and/or one or more digitalis alkaloids.

15. The one or more SGLT-2 inhibitors or pharmaceutically acceptable forms thereof for the use according to any one of claims 1 to 14, wherein the systolic blood pressure (SBP) value measured for the non-human mammal, preferably carnivore, more preferably a cat or a dog, in need thereof is decreased after the period of the treatment by at least 5 mmHg, preferably by at least 10 mmHg, more preferably by at least 20 mmHg, in particular by 5 to 100 mmHg, more preferably 5 to 50 mmHg, most preferably by 10 to 50 mmHg, in relation to the baseline SBP value measured for the non-human mammal, preferably carnivore, more preferably a cat or a dog, prior to the period of treatment.

16. The one or more SGLT-2 inhibitors or pharmaceutically acceptable forms thereof for the use according to any one of claims 1 to 15, wherein the method comprises measurement of the SBP and optionally identification of TOD followed by administration of a therapeutically effective amount of the one or more SGLT-2 inhibitors or pharmaceutically acceptable forms thereof to the non-human mammal in need thereof, preferably carnivore, more preferably a cat or a dog, wherein the therapeutically effective amount of the one or more SGLT-2 inhibitors or pharmaceutically acceptable forms thereof is administered in a daily dosage amount that may be varied over a treatment period depending on subsequent measurements of the SBP.

17. The one or more SGLT-2 inhibitors or pharmaceutically acceptable forms thereof for the use according to any one of claims 1 to 15, wherein the hypertension is non-refractory to the treatment with ACE inhibitors in the non-human mammal to be treated, preferably the carnivore to be treated, in particular the cat or dog to be treated, most preferably the dog to be treated.

18. A pharmaceutical composition comprising one or more SGLT-2 inhibitors or pharmaceutically acceptable forms thereof according to any one of claims 1 to 17 for use according to any one of claims 1 to 17.
